(19) United States
(12) Patent Application Publication
(10) Pub. No.: US 2010/0081938 A1
(43) Pub. Date: Apr. 1, 2010

(54) ULTRASONIC DIAGNOSTIC APPARATUS

(76) Inventor: Sei Kato, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/568,102

(22) Filed: Sep. 28, 2009

(30) Foreign Application Priority Data
Sep. 29, 2008 (JP) 2008-249608

(51) Int. Cl. A61B 8/14

(52) U.S. Cl. 600/458

(57) ABSTRACT

An ultrasonic diagnostic apparatus includes a display device for displaying an ultrasonic image of a subject has been given a contrast medium, a setting device for setting a region of interest in an ultrasonic image displayed in the display device, and a computing device for computing a time intensity curve indicating time change in the average brightness of the pixels in a set region of interest. The computing device excludes pixels corresponding to a non-observed object in a region of interest when computing a time intensity curve.

### CROSS REFERENCE TO RELATED APPLICATIONS

This application claims the benefit of Japanese Patent Application No. 2008-249608 filed Sep. 29, 2008, which is hereby incorporated by reference in its entirety.

### BACKGROUND OF THE INVENTION

The embodiments described herein relate to an ultrasonic diagnostic apparatus in which a time intensity curve is determined with respect to a region of interest set in an ultrasonic image.

In examinations using an ultrasonic diagnostic apparatus, a contrast medium may be injected into the body of a subject sometimes to enhance contrast to obtain a clearer image of a region to be diagnosed. In such examinations using a contrast medium, TIC (Time Intensity Curve) may be used sometimes. (Refer to Japanese Patent Application 2005-95376, for example.) TIC is a time intensity curve indicating time change in the average brightness in a region of interest (ROI) set on an ultrasonic image. Displaying this TIC makes it possible to observe change in the concentration of a contrast medium and to quantitatively grasp how a contrast medium is circulated in the body of a subject to diagnose the presence or absence of a disease in the subject or the severity of the disease.

The liver is one of internal organs to be diagnosed with an ultrasonic diagnostic apparatus. Description will be given to the functions of the liver. Nutriment absorbed through the stomach, small intestine, large intestine, or the like goes through the portal vein and enters the liver (tissue). The portal vein runs through hepatic cells and branches to thin blood vessels and conveys blood containing nutriment into every segment of the liver. The nutriment is taken into hepatic cells and resynthesized into a substance required for living bodies there. The resynthesized substance is returned into blood and enters hepatic vein and the large vein and it is then returned to the heart and conveyed to each part of the body.

To diagnose this liver, it is required to administer a contrast medium into a vein of an arm and continuously observe the following: how the blood flow containing the contrast medium osmoses from a thin blood vessel at the end of the hepatic portal into the hepatic tissue and is retained there.

However, what is indicated by TIC is time change in the average brightness of an entire ROI. Therefore, when ROI includes a blood vessel that is a region not observed by TIC in addition to the hepatic tissue that is a region to be observed by TIC, the following takes place: the brightness value indicated by TIC reflects both the contrast medium flowing through the blood vessel and the contrast medium osmosed into the hepatic tissue. For this reason, it is conventionally difficult to observe only how a contrast medium is osmosed into the hepatic tissue and retained there using TIC.

It is desirable that the problem described previously is solved.

### BRIEF DESCRIPTION OF THE INVENTION

A first aspect of the invention is an ultrasonic diagnostic apparatus including: a display device for displaying an ultrasonic image of a subject has been given a contrast medium; a setting device for setting a region of interest in an ultrasonic image displayed in the display device; and a computing device for computing a time intensity curve indicating time change in the average brightness of pixels in a set region of interest, wherein the computing device excludes the pixels corresponding to a non-observed object in a region of interest when computing a time intensity curve.

A second aspect of the invention is an ultrasonic diagnostic apparatus according to the first aspect of the invention including a display processing device, wherein the display processing device displays the pixels corresponding to a non-observed object in a region of interest in such a mode that they can be discriminated from the pixels corresponding to an observed object in the region of interest.

A third aspect of the invention is an ultrasonic diagnostic apparatus according to the first or second aspect of the invention, wherein the computing device identifies the pixels corresponding to a non-observed object on the basis of brightness and excludes these pixels when computing a time intensity curve.

A fourth aspect of the invention is an ultrasonic diagnostic apparatus according to the first or second aspect of the invention, wherein the computing device identifies the pixels corresponding to an observed object on the basis of brightness and computes a time intensity curve with respect to these pixels.

A fifth aspect of the invention is an ultrasonic diagnostic apparatus according to the third or fourth aspect of the invention including a brightness setting device, wherein the brightness setting device sets a brightness that provides a basis for identifying the pixels corresponding to a non-observed object or the pixels corresponding to an observed object.

A sixth aspect of the invention is an ultrasonic diagnostic apparatus according to any of the first to fifth aspects including: an adding device for carrying out weighting addition on a group of echo signals obtained by transmitting an ultrasonic wave to a subject has been given an ultrasonic contrast medium in an identical direction more than once; a filter for separating and extracting a fundamental component and a harmonic component from an output signal of the adding device; and a contrast-mode image generation device for generating a fundamental image pertaining to a fundamental component and a harmonic image pertaining to a harmonic component based on respective extracted signals of the filter, wherein the computing device identifies the pixels corresponding to a non-observed object or an observed object on the basis of brightness in either a fundamental image or a harmonic image when computing a time intensity curve.

A seventh aspect of the invention is an ultrasonic diagnostic apparatus according to the sixth aspect of the invention, wherein the computing device identifies pixels having a predetermined or higher brightness as the pixels corresponding to a non-observed object in an image with the non-observed object extracted of a fundamental image and a harmonic image; and it excludes these pixels when computing a time intensity curve.

An eighth aspect of the invention is an ultrasonic diagnostic apparatus according to the sixth aspect of the invention, wherein the computing device identifies pixels having a predetermined or lower brightness as the pixels corresponding to an observed object in an image with the non-observed object extracted of a fundamental image and a harmonic image; and it computes a time intensity curve with respect to these pixels.

A ninth aspect of the invention is an ultrasonic diagnostic apparatus according to any of the sixth to eighth aspects of the invention, wherein the contrast-mode image generation device generates an image obtained by combining a fundamental image and a harmonic image in such a mode that they can be discriminated from each other.

A tenth aspect of the invention is an ultrasonic diagnostic apparatus according to any of the first to fifth aspects of the invention including a B-mode image generation device. The B-mode image generation device generates a B-mode image based on an echo signal obtained by transmitting an ultrasonic wave to a subject has been given an ultrasonic contrast medium. The computing device identifies the pixels corresponding to a non-observed object or an observed object on the basis of brightness in a B-mode image when computing a time intensity curve.

An eleventh aspect of the invention is an ultrasonic diagnostic apparatus according to the tenth aspect of the invention including, in addition to the B-mode image generation device: an adding device for carrying out weighting addition on a group of echo signals obtained by transmitting an ultrasonic wave to a subject has been given an ultrasonic contrast medium in an identical direction more than once; a filter for separating and extracting a fundamental component and a harmonic component from an output signal of the adding device; a contrast-mode image generation device for generating a fundamental image pertaining to a fundamental component and a harmonic image pertaining to a harmonic component based on respective extracted signals of the filter; and a combination device for combining a fundamental image, a harmonic image, and a B-mode image, wherein the computing device identifies the pixels corresponding to a non-observed object or an observed object on the basis of brightness in any of a fundamental image, a harmonic image, and a B-mode image when computing a time intensity curve.

A twelfth aspect of the invention is an ultrasonic diagnostic apparatus according to any of the first to fifth aspects of the invention including a Doppler image generation device. The Doppler image generation device detects a Doppler signal based on an echo signal obtained by transmitting an ultrasonic wave to a subject has been given an ultrasonic contrast medium and generates a color Doppler image based on this Doppler signal. The computing device identifies the pixels corresponding to a non-observed object or an observed object on the basis of a flow velocity value or a power value in a color Doppler image when computing a time intensity curve.

A thirteenth aspect of the invention is an ultrasonic diagnostic apparatus according to the twelfth aspect of the invention including, in addition to the Doppler image generation device: an adding device for carrying out weighting addition on a group of echo signals obtained by transmitting an ultrasonic wave to a subject has been given an ultrasonic contrast medium in an identical direction more than once; a filter for separating and extracting a fundamental component and a harmonic component from an output signal of the adding device; a contrast-mode image generation device for generating a fundamental image pertaining to a fundamental component and a harmonic image pertaining to a harmonic component based on respective extracted signals of the filter; and a combination device for combining a fundamental image, a harmonic image, and a color Doppler image, wherein the computing device identifies the pixels corresponding to a non-observed object or an observed object on the basis of the following when computing a time intensity curve: brightness in a fundamental image or a harmonic image or a flow velocity value or a power value in a color Doppler image.

A fourteenth aspect of the invention is an ultrasonic diagnostic apparatus according to any of the first to fifth aspects of the invention including: an adding device for carrying out weighting addition on a group of echo signals obtained by transmitting an ultrasonic wave to a subject has been given an ultrasonic contrast medium in an identical direction more than once; a filter for separating and extracting a fundamental component and a harmonic component from an output signal of the adding device; a contrast-mode image generation device for generating a fundamental image pertaining to a fundamental component and a harmonic image pertaining to a harmonic component based on respective extracted signals of the filter; a B-mode image generation device for generating a B-mode image based on an echo signal obtained by transmitting an ultrasonic wave to a subject has been given an ultrasonic contrast medium; a Doppler image generation device for detecting a Doppler signal based on an echo signal obtained by transmitting an ultrasonic wave to a subject has been given an ultrasonic contrast medium and generating a color Doppler image based on this Doppler signal; and a combination device for combining a fundamental image, a harmonic image, a B-mode image, and a color Doppler image, wherein the computing device identifies the pixels corresponding to a non-observed object or an observed object based on the following when computing a time intensity curve: brightness in a fundamental image, a harmonic image, or a B-mode image or a flow velocity value or a power value in a color Doppler image.

A fifteenth aspect of the invention is an ultrasonic diagnostic apparatus according to any of the first to fifth aspects of the invention including a B-flow image generation device. The B-flow image generation device generates a B-flow image from an echo signal obtained by transmitting an ultrasonic wave to a subject has been given an ultrasonic contrast medium. The computing device identifies the pixels corresponding to a non-observed object or an observed object on the basis of brightness in a B-flow image when computing a time intensity curve.

A sixteenth aspect of the invention is an ultrasonic diagnostic apparatus according to the fifteenth aspect of the invention including, in addition to the B-flow image generation device: an adding device for carrying out weighting addition on a group of echo signals obtained by transmitting an ultrasonic wave to a subject has been given an ultrasonic contrast medium in an identical direction more than once; a filter for separating and extracting a fundamental component and a harmonic component from an output signal of the adding device; a contrast-mode image generation device for generating a fundamental image pertaining to a fundamental component and a harmonic image pertaining to a harmonic component based on respective extracted signals of the filter; and a combination device for combining a fundamental image, a harmonic image, and a B-flow image. The computing device identifies the pixels corresponding to a non-observed object or an observed object on the basis of brightness in any of a fundamental image, a harmonic image, and a B-flow image when computing a time intensity curve.

A seventeenth aspect of the invention is an ultrasonic diagnostic apparatus according to any of the first to sixteenth aspects of the invention, wherein a non-observed object is blood flow.

An eighteenth aspect of the invention is an ultrasonic diagnostic apparatus according to the seventeenth aspect of the invention, wherein a fundamental image pertaining to a fundamental component extracted from the filter is an image obtained by extracting a blood flow portion; and the computing device excludes pixels having a predetermined or higher brightness in the image obtained by extracting a blood flow portion when computing a time intensity curve.

A nineteenth aspect of the invention is an ultrasonic diagnostic apparatus including: a display device for displaying an ultrasonic image of a subject has been given a contrast medium; a setting device for setting a region of interest in an ultrasonic image displayed in the display device; a computing device for computing a time intensity curve indicating time change in the average brightness of pixels in a set region of interest; and a contrast-mode image generation device for generating a contrast-mode image as an ultrasonic image to be displayed in the display device based on a signal with a frequency component obtained from a non-observed object excluded of echo signals obtained by transmitting an ultrasonic wave to a subject.

A twentieth aspect of the invention is an ultrasonic diagnostic apparatus according to the nineteenth aspect of the invention, wherein a non-observed object is blood flow and the computing device computes a time intensity curve with respect to a contrast-mode image generated based on a signal with a fundamental component obtained from a blood flow portion excluded of echo signals.

According to the embodiments described herein, the computing device excludes the pixels corresponding to a non-observed object when computing the average brightness in a region of interest and computes a time intensity curve indicating time change therein. Therefore, a time intensity curve can be obtained only with respect to an observed object.

According to the embodiments described herein, the computing device computes a time intensity curve in a region of interest set in a contrast-mode image generated based on a signal with a frequency component obtained from a non-observed object excluded of echo signals. Therefore, a time intensity curve can be obtained only with respect to a region to be observed.

Further objects and advantages of the embodiments described herein will be apparent from the following description as illustrated in the accompanying drawings.

### BRIEF DESCRIPTION OF THE DRAWINGS

FIG. 1 is a block diagram illustrating the configuration of an ultrasonic diagnostic apparatus in an example of embodiments of the invention.

FIG. 2 is a block diagram illustrating the configuration of the contrast-mode processing unit in the ultrasonic diagnostic apparatus in FIG. 1.

FIG. 3 is a block diagram illustrating the configuration of the image processing unit in the ultrasonic diagnostic apparatus illustrated in FIG. 1.

FIG. 4 is a schematic diagram illustrating a signal processing system used in ultrasonic imaging in the first embodiment.

FIGS. 5(A) and 5(B) are drawings illustrating an example of ultrasonic images displayed in a display unit.

FIG. 6 is a flowchart illustrating TIC computation.

FIG. 7 is a drawing illustrating an example of ROI set in an ultrasonic image.

FIG. 8 is a drawing illustrating the TIC of the portion of the hepatic tissue as the observed object in the ROI illustrated in FIG. 7.

FIG. 9 is a schematic diagram illustrating signal processing systems in ultrasonic imaging in a second embodiment.

FIG. 10 is a schematic diagram illustrating signal processing systems and the like in ultrasonic imaging in a third embodiment.

FIG. 11 is a drawing illustrating a signal processing system in ultrasonic imaging in a fourth embodiment.

### DETAILED DESCRIPTION OF THE INVENTION

Hereafter, description will be given to embodiments of the invention with reference to drawings.

### First Embodiment

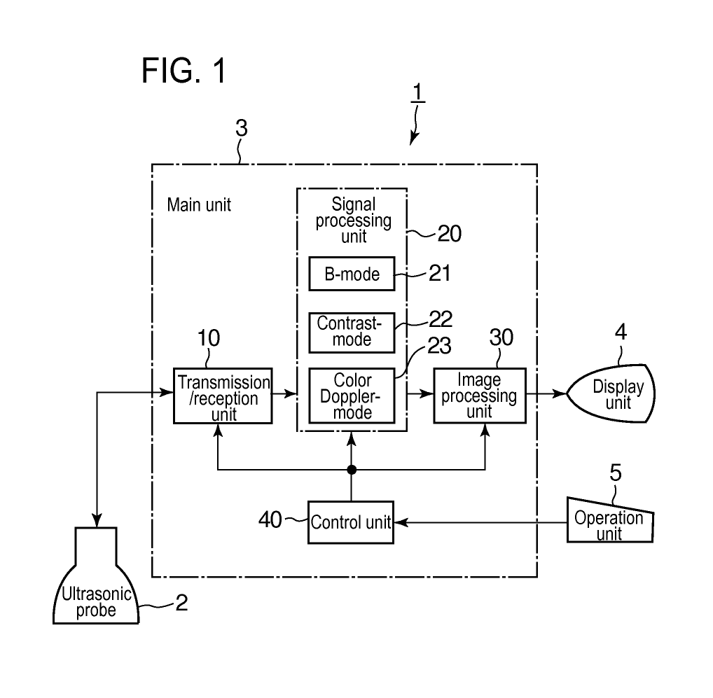

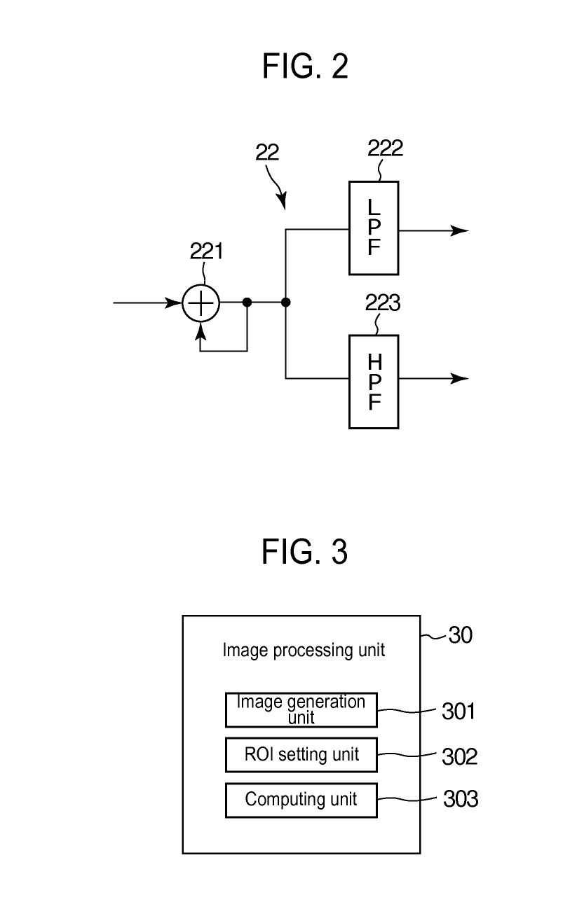

First, description will be given to a first embodiment of the invention. FIG. 1 is a block diagram illustrating the configuration of an ultrasonic diagnostic apparatus in an example of embodiments of the invention; FIG. 2 is a block diagram illustrating the configuration of the contrast-mode processing unit in the ultrasonic diagnostic apparatus illustrated in FIG. 1; and FIG. 3 is a block diagram illustrating the configuration of the image processing unit in the ultrasonic diagnostic apparatus illustrated in FIG. 1.

The ultrasonic diagnostic apparatus 1 in this example includes: an ultrasonic probe 2 that transmits and receives ultrasonic waves; an apparatus main unit 3; a display unit 4 that displays an ultrasonic image and the like; and an operation unit 5 including a keyboard, a pointing device, and the like. The display unit 4 is an example of display device in embodiments of the invention.

The apparatus main unit 3 includes: a transmission/reception unit 10 that drives the ultrasonic probe 2 to transmit and receive an ultrasonic wave to and from a subject; a signal processing unit 20 that carries out signal processing according to various image display modes based on echo signals from a subject; an image processing unit 30 that generates ultrasonic images corresponding to various image display modes and displays them in the display unit 4; and a control unit 40 that carries out main control and processing of the ultrasonic diagnostic apparatus 1. The signal processing unit 20 includes a B-mode processing unit 21, a contrast-mode processing unit 22, and a color Doppler-mode processing unit 23. One of or a combination of two or more of the individual mode processing units 21 to 23 is used according to the purpose of ultrasonic diagnosis.

Though the detailed configuration thereof is not shown in drawings, the B-mode processing unit 21 is so constructed that the following is implemented: it carries out processing, such as amplification, logarithmic compression, and envelope detection, on echo signals from the transmission/reception unit 10 to generate B-mode data. The B-mode processing unit 21 and the image generation unit 301, described later, of the image processing unit 30 are an example of B-mode image generation device in embodiments of the invention.

The contrast-mode processing unit 22 includes: an adding unit 221 that carries out weighting addition on echo signals obtained by transmitting an ultrasonic wave to a subject in an identical direction more than once as described later; a low pass filter (LPF) 222 that extracts a fundamental component from an addition signal; and a high pass filter (HPF) 223 that extracts a harmonic component from an addition signal. The low pass filter 222 and the high pass filter 223 are an example of filter in embodiments of the invention.

As described later, a fundamental image is generated based on the signal of a fundamental component extracted through the low pass filter 222 and a harmonic image is generated based on the signal of a harmonic component extracted through the high pass filter 223. The fundamental image and the harmonic image are combined at the image generation unit 301 and a contrast-mode image is generated. The contrast-mode processing unit 22 and the image generation unit 301 are an example of contrast-mode image generation device in embodiments of the invention.

Though the detailed configuration thereof is not shown in drawings, the color Doppler-mode processing unit 23 is so constructed that the following is implemented: it carries out processing, such as MTI (Moving Target Indication), autocorrelation, and velocity/variance/power computation, on echo signals from the transmission/reception unit 10 to determine velocity, variance, and power. The image generation unit 301 generates color Doppler images based on the respective signals obtained as the result of these types of processing. The color Doppler-mode processing unit 23 and the image generation unit 301 are an example of Doppler image generation device in embodiments of the invention. Incidentally, the color Doppler image includes CFM (Color Flow Mapping) image, power Dopper image, and variance image.

As illustrated in FIG. 3, the image processing unit 30 includes: the image generation unit 301 that generates ultrasonic images corresponding to the various image display modes; an ROI setting unit 302 that sets ROI in an ultrasonic image; and a computing unit 303 that computes TIC indicating time change in average brightness in ROI. The ROI setting unit 302 is an example of setting device in embodiments of the invention and the computing unit 303 is an example of computing device in embodiments of the invention.

The computing unit 303 excludes the pixels corresponding to a non-observed object in ROI when computing TIC. When computing TIC, the computing unit 303 identifies the pixels corresponding to a non-observed object on the basis of brightness. In this example, it identifies pixels having a predetermined or higher brightness value as the pixels corresponding to a non-observed object.

Incidentally, in the description of embodiments of the invention, contrast imaging of a liver will be taken as an example and the observed object is tissue portion. The non-observed object is blood flow portion. The image generation unit 301 generates the following image as an image to be displayed in the display unit 4: an image displayed in such a mode that the pixels corresponding to the blood flow portions can be discriminated from the pixels corresponding to the tissue portions. In this example, the image generation unit 301 generates images in which blood flow portions and tissue portions are displayed in different colors, as described later. The image generation unit 301 is an example of display processing device in embodiments of the invention.

The computing unit 303 identifies the pixels corresponding to a non-observed object in an image with the non-observed object extracted of the following images generated at the image generation unit 301, as described later: a fundamental image generated based on the signal of a fundamental component and a harmonic image generated based on the signal of a harmonic component. As mentioned above, the non-observed object is blood flow portions. Of the fundamental image and the harmonic image, an image with blood flow portions mainly extracted is the fundamental image. Therefore, the computing unit 303 identifies pixels having a predetermined or higher brightness value as the pixels corresponding to the blood flow portions in the fundamental image.

Incidentally, a brightness value that provides a threshold value for identifying the pixels corresponding to a non-observed object can be set by the operation unit 5. The operation unit 5 is an example of setting device in embodiments of the invention.

Description will be given to ultrasonic imaging using an ultrasonic diagnostic apparatus 1 of the invention. In this description, a case where an image of a subject is captured only in contrast-mode will be taken as an example.

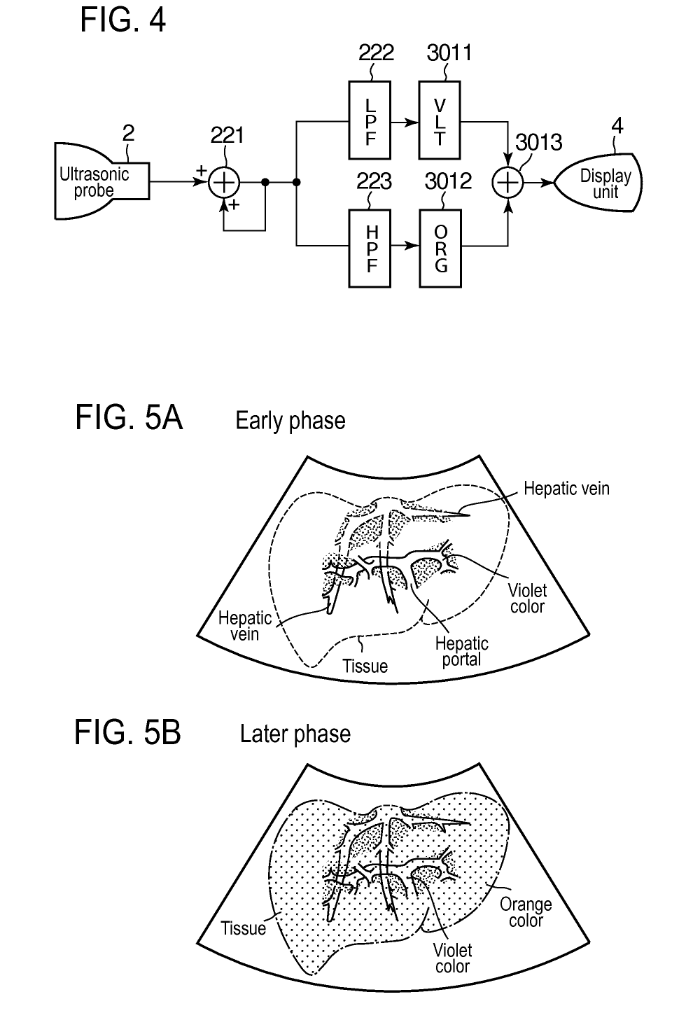

FIG. 4 is a schematic diagram illustrating a signal processing system used in the ultrasonic imaging in the example. FIG. 4 depicts only the outline of signal processing. First, the transmission/reception unit 10 (not shown in FIG. 4) alternately transmits two ultrasonic pulse signals P1, P2 (not shown) with the phase thereof inverted from the ultrasonic probe 2. An echo signal E1 to the ultrasonic pulse signal P1 and an echo signal E2 to the ultrasonic pulse signal P2 are added at the adding unit 221 of the contrast-mode processing unit 22. The addition signal of the echo signal E1 and the echo signal E2 is processed through the low pass filter 222 and the high pass filter 223.

Description will be given to the addition signal of the echo signal E1 and the echo signal E2. In the tissue portion where the blood flow rate is low, the harmonic component of the addition signal is predominant. Meanwhile, in the blood flow portion where the blood flow rate is high, many fundamental components of the addition signal are contained. (Refer to JP-A-2004-147823.) Therefore, the signals of fundamental components extracted from the low pass filter 222 are mainly signals pertaining to blood flow portions. Meanwhile, the signals of harmonic components extracted from the high pass filter 223 are mainly signals pertaining to tissue portions.

Signals from the low pass filter 222 and signals from the high pass filter 223 are outputted to the image processing unit 30 (not shown in FIG. 4). At the image generation unit 301 (not shown in FIG. 4) of the image processing unit 30, the following takes place: a fundamental image is generated based on a signal from the low pass filter 222 and a harmonic image is generated based on a signal from the high pass filter 223. Specific description will be given. The image generation unit 301 adds data of violet color to the fundamental components of signals from the low pass filter 222 at a violet data adding unit (VLT) 3011. Thus it displays mainly blood flow portions in violet and generates a fundamental image that is an image in which mainly blood flow portions are extracted. The image generation unit 301 adds data of orange color to the harmonic components of signals from the high pass filter 223 at an orange data adding unit (ORG) 3012. Thus it displays mainly tissue portions in orange and generates a harmonic image that is an image in which mainly tissue portions are extracted. The image generation unit 301 combines the fundamental image and the harmonic image at a combination unit 3013 to generate a contrast-mode image and displays this contrast-mode image in the display unit 4 as an ultrasonic image.

FIGS. 5(A) and 5(B) illustrate examples of ultrasonic images displayed in the display unit 4. FIG. 5(A) illustrates an example of an ultrasonic image in the early phase of contrast imaging; and FIG. 5(B) illustrates an example of an ultrasonic image in the later phase of contrast imaging. Incidentally, FIG. 5(B) depicts an ultrasonic image in the later phase at t=1 to 2 minutes or so.

In the early phase, as illustrated in FIG. 5(A), it is observed how a contrast medium administered into a portal vein is conveyed together with a blood flow to thin blood vessels at the end thereof. The blood from thin blood vessels at the end of the portal vein starts to be retained in the hepatic tissue in the boundary area and the area is colored orange. At the same time, part of the blood flow is bypassed from the end of the portal vein to the ends of hepatic veins. In this example, only the contrast-mode image is observed; therefore, the overall shape of the liver is not displayed.

In the later phase, as illustrated in FIG. 5(B), the blood from the portal vein is being retained in substantially the entire hepatic tissue and a wider area in the liver is colored orange. When the contrast medium continuously flows into the hepatic portal, the blood flow portions are continuously colored violet. Therefore, the blood flow portions and the tissue containing a tumor or the like therearound can be easily discriminated from each other even in the later phase of the observation.

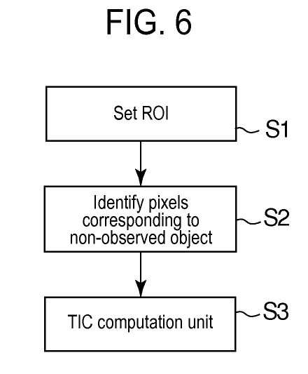

Description will be given to the computation of TIC. FIG. 6 is a flowchart illustrating TIC computation. At Step S1, first, ROI is set in the ultrasonic image displayed in the display unit 4. The ROI is set by the ROI setting unit 302 based on a signal from the operation unit 5.

After the ROI is set at Step S1, the computing unit 303 carries out the following processing at Step S2: it identifies pixels whose brightness value of violets is equal to or higher than a predetermined brightness value in the portion equivalent to the ROI in the fundamental image before combined at the combination unit 3013. These pixels correspond to blood flow portions excluded from TIC computation. At Step S3, the computing unit 303 computes the average brightness of the pixels excluding the pixels corresponding to the blood flow portions with respect to the synthetic image. This computation of average brightness is periodically carried out. The result of computation is displayed in the display unit 4 as TIC indicating time change in the average brightness.

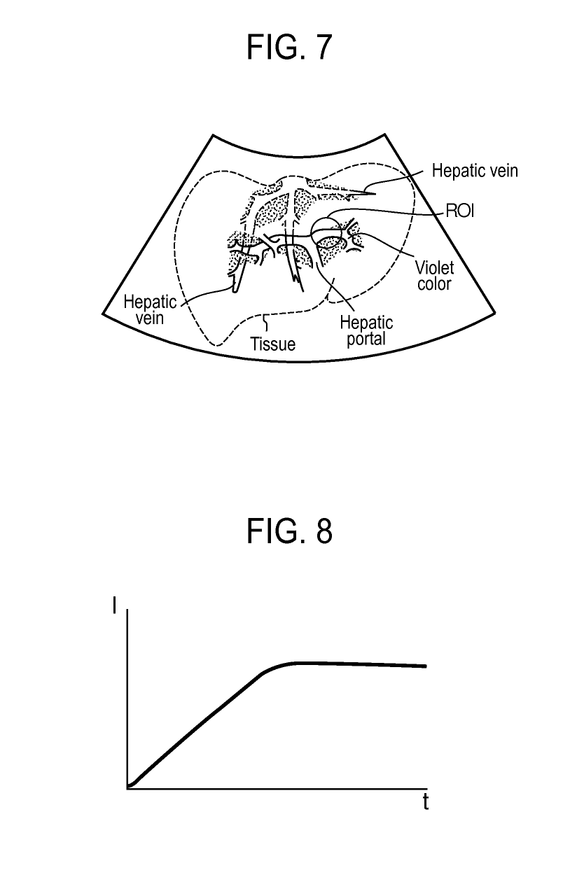

FIG. 7 illustrates an example of ROI set in an ultrasonic image (contrast-mode image). In the example in FIG. 7, the ROI is so set that the hepatic portal and the hepatic tissue are embraced. FIG. 8 illustrates the TIC of the portion of the hepatic tissue as the observed object in the ROI illustrated in FIG. 7. The TIC is computed at the computing unit 303. As illustrated in FIG. 7, the TIC is computed with the blood flow portions as the non-observed object excluded even when the ROI embraces the hepatic portal as a blood flow portion. Therefore, the TIC illustrated in FIG. 8 accurately represents time change in the average brightness of only the hepatic tissue as the observed object.

According to this embodiment described up to this point, the computing unit 303 excludes the pixels corresponding to the blood flow portions as non-observed objects when computing the average brightness in the ROI and computes the TIC. Therefore, the TIC can be obtained only with respect to the hepatic tissue as the observed object.

The pixels corresponding to the blood flow portions as non-observed objects are displayed in violet. That is, they are displayed in color different from the color of the pixels corresponding to the hepatic tissue as the object observed by the TIC. Therefore, the non-observed objects can be displayed in such a mode that they can be discriminated from the observed object.

### Second Embodiment

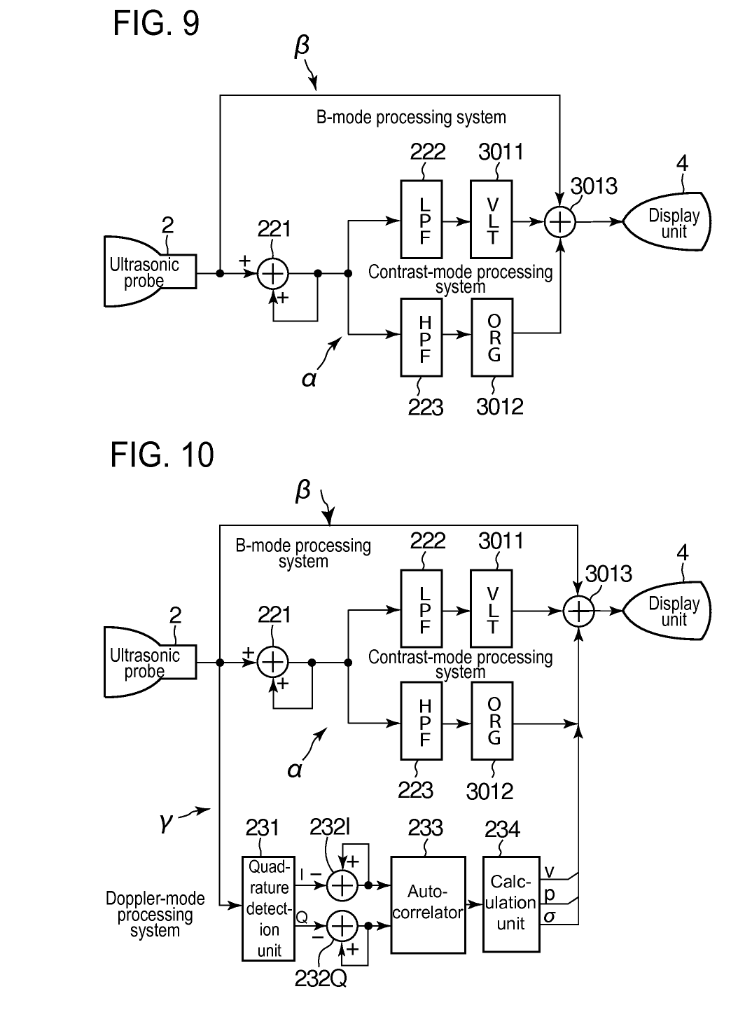

Description will be given to a second embodiment of the invention. FIG. 9 is a schematic diagram illustrating signal processing systems in ultrasonic imaging in the second embodiment. In the second embodiment, an ordinary B-mode image is displayed over a contrast-mode image in the above-mentioned contrast mode. In FIG. 9, a denotes a signal processing system for contrast mode and β denotes a signal processing system for B mode. With the signal processing system a for contrast mode, a fundamental image and a harmonic image are obtained similarly with the foregoing. With the signal processing system β for B mode, a monochrome B-mode image is obtained. A fundamental image, a harmonic image, and a B-mode image obtained with the individual signal processing systems α, 13 are combined at the combination unit 3013 and the ultrasonic image obtained as the result of this synthesis is displayed in the display unit 4. Since this synthetic image includes a B-mode image, it is possible to observe the entire tissue of the liver even in the early phase of observation.

In this embodiment, the computing unit 303 carries out the following processing when computing the TIC of ROI set in a synthetic image displayed in the display unit 4: it identifies the pixels corresponding to a blood flow portion as a non-observed object in either of a fundamental image and a B-mode image before combined at the combination unit 3013. When the pixels are identified in the fundamental image, the pixels whose brightness value of violets is equal to or higher than a predetermined brightness value are identified as the pixels corresponding to blood flow portions as mentioned above. In B-mode images, meanwhile, blood flow portions are lower in brightness value than tissue portions. When the pixels are identified in a B-mode image, therefore, the pixels whose brightness value of blacks and whites is equal to or lower than a predetermined brightness value are identified as the pixels corresponding to blood flow portions.

Also in this embodiment described up to this point, the same effect as in the first embodiment can be obtained.

### Third Embodiment

Description will be given to a third embodiment of the invention. FIG. 10 is a schematic diagram illustrating signal processing systems and the like in ultrasonic imaging in the third embodiment. In the third embodiment, an image of blood flow information in color Doppler mode is also overlaid and displayed in addition to a contrast-mode image in contrast mode and an ordinary B-mode image. In FIG. 10, α denotes a signal processing system for contrast mode; β denotes a signal processing system for B mode; and γ denotes a signal processing system for color Doppler mode. A publicly known system can be used for the signal processing system γ for color Doppler mode and the drawing illustrates the configuration of an example of such a system. In the signal processing system γ for color Doppler mode, the color Doppler-mode processing unit 23 includes: a quadrature detection unit 231 that carries out reception and detection on the orthogonal I- and Q-axis components of multiple echo signals without the reversal of polarity; subtracters 232I, 232Q that determine the differences between the respective detected outputs I1, I2 and Q1, Q2 equivalent to at least two prior and subsequent waves; an autocorrelator 233 that carries out frequency analysis by the computation of periods or the like at high speed based on the I- and Q-signals of the respective subtraction outputs; and a calculation unit 234 that obtains blood flow information, including flow velocity v, power p, and the variance σ of flow velocity, and the like, from the result of frequency analysis. The color Doppler images obtained with the signal processing system γ for color Doppler mode are images described below. For example, when they are images with flow velocity and variance combined together, the blood flow toward the ultrasonic probe 2 is displayed in red and the blood flow away therefrom is displayed in blue; the velocity is represented by brightness; and the variance is represented by the mixture of green. When they are color Doppler images in which the power is imaged, the brightness of display colors of the images corresponds to power.

A fundamental image, a harmonic image, a B-mode image, and a color Doppler image obtained with the individual signal processing systems α, β, γ are combined at the combination unit 3013 and the ultrasonic image obtained as the result of this synthesis is displayed in the display unit 4.

In this embodiment, the computing unit 303 carries out the following processing when computing the TIC of ROI set in a synthetic image displayed in the display unit 4: it identifies the pixels corresponding to a blood flow portion as a non-observed object of the TIC in any of the following: a fundamental image, a B-mode image, and a color Doppler image before combined at the combination unit 3013. When they are identified in a fundamental image or a B-mode image, the same processing as in the second embodiment and the description thereof will be omitted. When they are identified in a color Doppler image, pixels having a predetermined or higher flow velocity value or a predetermined or higher power value are identified as the pixels corresponding to blood flow portions.

Also in this embodiment described up to this point, the same effect as in the first and second embodiments can be obtained.

### Fourth Embodiment

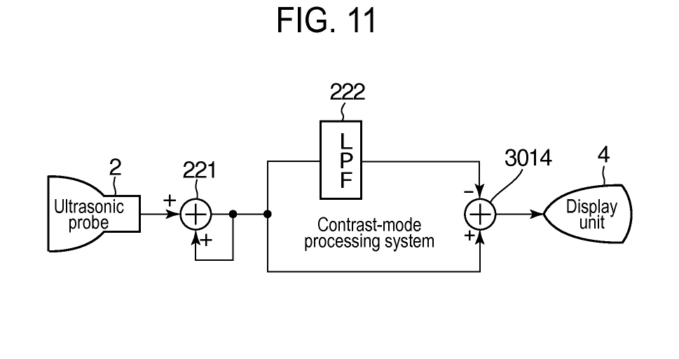

Description will be given to a fourth embodiment of the invention. FIG. 11 illustrates a signal processing system in ultrasonic imaging in the fourth embodiment. In this embodiment, the contrast-mode processing unit 22 does not have a high pass filter. More specific description will be given. In the contrast-mode processing unit 22, the system for processing signals added at the adding unit 221 is divided into two systems. In one system, signals are processed through the low pass filter 222 and then outputted to the image processing unit 30. In the other system, signals are directly outputted to the image processing unit 30. As a result, the following signals are inputted to the image processing unit 30: the signals of fundamental components that went through the low pass filter 222 and signals containing both fundamental components and harmonic components that did not go through a filter.

The image generation unit 301 of the image processing unit 30 subtracts the signals of fundamental components from signals containing both fundamental components and harmonic components by a subtracter 3014. Then the image generation unit 301 generates a contrast-mode image from a signal obtained at the subtracter 3014 and displays this image in the display unit 4 as an ultrasonic image. In the obtained ultrasonic image, incidentally, the brightness of blood flow portions is lower than the brightness of tissue portions.

In this embodiment, the computing unit 303 averages the brightness values of all the pixels in ROI when computing the TIC of the ROI set in an ultrasonic image displayed in the display unit 4. The ultrasonic images displayed in the display unit 4 are images generated based on the following signals: signals obtained by subtracting the signals of fundamental components from signals containing both fundamental components and harmonic components, that is, signals with a frequency component obtained mainly from a blood flow portion as a non-observed object excluded. Since TIC in ROI set in such an image is computed, therefore, it is possible to obtain TIC only with respect to the tissue portion as a region to be observed.

Up to this point, description has been given to the invention based on the above-mentioned embodiments. However, the invention can be variously modified without departing from the subject matter of the invention, needless to add. For example, the following image may be used as an image for identifying the pixels corresponding to a non-observed object of TIC: a B-flow image indicating flow information obtained by carrying out MTI processing, autocorrelation, and velocity/variance/power computation power processing on echo signals obtained by transmitting an ultrasonic wave to a subject.

The generated ultrasonic images are not limited to those in the above-mentioned embodiments. For example, an ultrasonic image obtained by combining a contrast-mode image and a color Doppler image or the like may be generated.

The computing unit 303 may carry out the following processing to compute TIC: pixels having a predetermined or lower brightness are identified as the pixels corresponding to a tissue portion as the observed object in a fundamental image and the average value of these pixels is computed.

The computing unit 303 may carry out the following processing to compute TIC depending on the observed object: the pixels corresponding to a non-observed object or an observed object are identified on the basis of brightness in a harmonic image.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a display device configured to display an ultrasonic image of a subject that has been given a contrast medium;
a setting device configured to set a region of interest in the ultrasonic image displayed by said display device; and
a computing device configured to compute a time intensity curve indicating time change in an average brightness of pixels in the set region of interest, wherein said computing device is further configured to exclude pixels corresponding to a non-observed object in the region of interest when computing the time intensity curve.

2. The ultrasonic diagnostic apparatus according to claim 1, further comprising:
a display processing device configured to display the pixels corresponding to the non-observed object in the region of interest such that the pixels can be discriminated from pixels corresponding to an observed object in the region of interest.

3. The ultrasonic diagnostic apparatus according to claim 1, wherein said computing device is further configured to:
identify the pixels corresponding to the non-observed object based on a brightness; and
exclude these pixels when computing the time intensity curve.

4. The ultrasonic diagnostic apparatus according to claim 1, wherein said computing device is configured to:
identify pixels corresponding to an observed object based on a brightness; and
compute the time intensity curve with respect to these pixels.

5. The ultrasonic diagnostic apparatus according to claim 3, further comprising:
a brightness setting device configured to set a brightness that provides a basis for identifying one of the pixels corresponding to a non-observed object and pixels corresponding to an observed object.

6. The ultrasonic diagnostic apparatus according to claim 1, further comprising:
an adding device configured to perform weighting addition on a group of echo signals obtained by transmitting a plurality of ultrasonic waves in an identical direction to the subject;
a filter configured to separate and extract a fundamental component and a harmonic component from an output signal of said adding device; and
a contrast-mode image generation device configured to generate a fundamental image pertaining to the fundamental component and a harmonic image pertaining to the harmonic component based on respective extracted signals of said filter, wherein:
said computing device identifies is configured to identify one of the pixels corresponding to the non-observed object and pixels corresponding to an observed object based on a brightness in either the fundamental image or the harmonic image when computing the time intensity curve.

7. The ultrasonic diagnostic apparatus according to claim 6, wherein said computing device is configured to:
identify one of pixels having a brightness greater than or equal to a predetermined brightness as the pixels corresponding to the non-observed object in an image with the non-observed object extracted of the fundamental image and the harmonic image; and
exclude these pixels when computing the time intensity curve.

8. The ultrasonic diagnostic apparatus according to claim 6, wherein said computing device is configured to:
identify pixels having a brightness less than or equal to a predetermined brightness as the pixels corresponding to the observed object in an image with the non-observed object extracted of the fundamental image and the harmonic image; and
compute the time intensity curve with respect to these pixels.

9. The ultrasonic diagnostic apparatus according to claim 6, wherein said contrast-mode image generation device is configured to generate an image obtained by combining the fundamental image and the harmonic image such that these images can be discriminated from each other.

10. The ultrasonic diagnostic apparatus according to claim 1, further comprising:
a B-mode image generation device configured to generate a B-mode image based on an echo signal obtained by transmitting the ultrasonic wave to the subject, wherein said computing device is configured to identify one of the pixels corresponding to the non-observed object and pixels corresponding to an observed object based on a brightness in the B-mode image when computing the time intensity curve.

11. The ultrasonic diagnostic apparatus according to claim 10, further comprising:
an adding device configured to perform weighting addition on a group of echo signals obtained by transmitting a plurality of ultrasonic waves in an identical direction to the subject;
a filter configured to separate and extract a fundamental component and a harmonic component from an output signal of said adding device;
a contrast-mode image generation device configured to generate a fundamental image pertaining to the fundamental component and a harmonic image pertaining to the harmonic component based on respective extracted signals of said filter; and
a combination device a configured to combine the fundamental image, the harmonic image, and the B-mode image, wherein said computing device is further configured to identify one of the pixels corresponding to the non-observed object and the pixels corresponding to an observed object based on a brightness in any of the fundamental image, the harmonic image, and the B-mode image when computing the time intensity curve.

12. The ultrasonic diagnostic apparatus according to claim 1, further comprising:
a Doppler image generation device configured to:
detect a Doppler signal based on an echo signal obtained by transmitting the ultrasonic wave to the subject; and
generating generate a color Doppler image based on this Doppler signal, wherein said computing device is further configured to identify one of the pixels corresponding to the non-observed object and pixels corresponding to an observed object based on one of a flow velocity value and a power value in the color Doppler image when computing the time intensity curve.

13. The ultrasonic diagnostic apparatus according to claim 12, further comprising:
an adding device configured to perform weighting addition on a group of echo signals obtained by transmitting a plurality of ultrasonic waves in an identical direction to the subject;
a filter configured to separate and extract a fundamental component and a harmonic component from an output signal of said adding device;
a contrast-mode image generation device configured to generate a fundamental image pertaining to the fundamental component and a harmonic image pertaining to the harmonic component based on respective extracted signals of said filter; and
a combination device configured to combine the fundamental image, the harmonic image, and the color Doppler image, wherein said computing device is further configured to identify one of the pixels corresponding to the non-observed object and the pixels corresponding to an observed object based on a brightness in one of the fundamental image, the harmonic image, the flow velocity value, and the power value in the color Doppler image when computing the time intensity curve.

14. The ultrasonic diagnostic apparatus according to claim 1, further comprising:
an adding device configured to perform weighting addition on a group of echo signals obtained by transmitting a plurality of ultrasonic waves in an identical direction to the subject;
a filter configured to separate and extract a fundamental component and a harmonic component from an output signal of said adding device;
a contrast-mode image generation device configured to generate a fundamental image pertaining to the fundamental component and a harmonic image pertaining to the harmonic component based on respective extracted signals of said filter;
a B-mode image generation device configured to generate a B-mode image based on an echo signal obtained by transmitting the ultrasonic wave to the subject;
a Doppler image generation device configured to:
detect a Doppler signal based on an echo signal obtained by transmitting the ultrasonic wave to the subject; and
generate a color Doppler image based on this Doppler signal; and
a combination device configured to combine the fundamental image, the harmonic image, the B-mode image, and the color Doppler image, wherein said computing device is configured to identify one of the pixels corresponding to the non-observed object and pixels corresponding to an observed object based on one of a brightness in the fundamental image, the harmonic image, and the B-mode image, and a flow velocity value and a power value in the color Doppler image when computing the time intensity curve.

15. The ultrasonic diagnostic apparatus according to claim 1, further comprising:
a B-flow image generation device configured to generate a B-flow image from an echo signal obtained by transmitting the ultrasonic wave to the subject, wherein said computing device is configured to identify one of the pixels corresponding to the non-observed object and pixels corresponding to an observed object based on of a brightness in the B-flow image when computing the time intensity curve.

16. The ultrasonic diagnostic apparatus according to claim 15, further comprising:
an adding device configured to perform weighting addition on a group of echo signals obtained by transmitting a plurality of ultrasonic waves in an identical direction to the subject;
a filter configured to separate and extract a fundamental component and a harmonic component from an output signal of said adding device;
a contrast-mode image generation device configured to generate a fundamental image pertaining to the fundamental component and a harmonic image pertaining to the harmonic component based on respective extracted signals of said filter; and
a combination device configured to combine the fundamental image, the harmonic image, and the B-flow image, wherein said computing device is further configured to identify one of the pixels corresponding to the non-observed object and pixels corresponding to an observed object based on a brightness in any of the fundamental image, the harmonic image, and the B-flow image when computing the time intensity curve.

17. The ultrasonic diagnostic apparatus according to claim 1, wherein the non-observed object is blood flow.

18. The ultrasonic diagnostic apparatus according to claim 17, wherein a fundamental image pertaining to a fundamental component extracted from a filter is an image obtained by extracting a blood flow portion and, wherein said computing device is further configured to exclude pixels having a brightness greater than or equal to a predetermined brightness in an image obtained by extracting the blood flow portion when computing the time intensity curve.

19. An ultrasonic diagnostic apparatus comprising:
a display device configured to display an ultrasonic image of a subject that has been given a contrast medium;
a setting device configured to set a region of interest in the ultrasonic image;
a computing device configured to compute a time intensity curve indicating time change in an average brightness of pixels in the set region of interest; and
a contrast-mode image generation device configured to generate a contrast-mode image as the ultrasonic image to be displayed on said display device based on a signal with a frequency component obtained from a non-observed object excluded of echo signals obtained by transmitting an ultrasonic wave to the subject.

20. The ultrasonic diagnostic apparatus according to claim 19, wherein the non-observed object is blood flow and said computing device is further configured to compute a time intensity curve with respect to the contrast-mode image generated based on the signal with a fundamental component obtained from the blood flow portion excluded of echo signals.
